(19) United States
(12) Patent Application Publication
(10) Pub. No.: US 2012/0276019 A1
(43) Pub. Date: Nov. 1, 2012

(54) TISSUE KALLIKREIN FOR THE TREATMENT OF PARKINSON'S DISEASE

(75) Inventors: Matthew L. Charles, Winnipeg (CA); Mark S. Williams, Winnipeg (CA)

(73) Assignee: DIAMEDICA INC., Winnipeg (CA)

(21) Appl. No.: 13/494,788

(22) Filed: Jun. 12, 2012

Related U.S. Application Data
(63) Continuation-in-part of application No. 13/055,660, filed on Mar. 4, 2011.
(60) Provisional application No. 61/083,650, filed on Jul. 25, 2008.

(51) Int. Cl. A61K 38/48; A61P 25/16; A61N 1/05; A61P 25/00; A61P 25/24; A61K 9/12; A61P 25/28

(52) U.S. Cl. 424/43; 424/94.64; 424/94.5; 607/116

(57) ABSTRACT

Provided herein are methods of treating Parkinson's disease, dementia with Lewy bodies, and conditions associated with Parkinson's disease and dementia with Lewy bodies. These methods include administering to a subject in need thereof a therapeutically effective amount of a tissue kallikrein (KLK1) polypeptide, including active variants and fragments thereof.

### CROSS-REFERENCE TO RELATED APPLICATIONS

This application is a continuation-in-part of U.S. application Ser. No. 13/055,660, filed Mar. 4, 2011, which is a U.S. National Stage Entry of PCT/CA2009/001051, filed Jul. 24, 2009, which claims benefit under 35 U.S.C. §119(e) of U.S. Application No. 60/083,650, filed Jul. 25, 2008; each of which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is DIAM_{—}019_{—}02US_ST25.txt. The text file is 8 KB, was created on Jun. 12, 2012, and is being submitted electronically via EFS-Web.

### FIELD OF THE INVENTION

The present invention relates to methods of treating Parkinson's disease, dementia with Lewy Bodies, and other conditions associated therewith.

### DESCRIPTION OF THE RELATED ART

Parkinson's disease (PD) is a degenerative condition of the central nervous system and the second most common neurodegenerative disease. PD is known to affect individuals over 60 and 80 years of age at rates of >1% and up to 4%, respectively (Schapira et al., Lancet Neurol, 2008, 7: 97-109). PD is typically characterized by the loss of dopaminergic neurons in particular regions of the brain, namely the substantia nigra pars compacta (SNc) (Marras et al., Neurology, 2008, 70(21):1996-2003).

This hindered neurotransmission results in tremor, bradykinesia, and muscular rigidity (Lee, J Mol Neurosci, 2008, 34: 17-22). PD and dementia with Lewy bodies (DLB) can be characterized by the presence of intracytoplasmic inclusions known as Lewy bodies (LB). LB are seen to have a somewhat filamentous structure in which immunohistochemical analysis has revealed that α-synuclein (α-Syn) may be the major component of LB in PD and DLB (Suh et al., Pharmacological Reviews, 2002, 54(3): 469-525). Lewy neurites are similar structures to Lewy bodies and are composed of α-Syn. However, lewy neurites are localized in the axon or dendrite projections of a neuron. These lewy neurites can progress to other regions of the brain and may be responsible for secondary symptoms of PD. Secondary symptoms of PD include depression, dementia, insomnia, autonomic and/or sensory dysfunction, among others (Lee, 2008).

Dementia with Lewy Bodies (DLB) is the second most common form of dementia (Neef, et al., Am Fam Physician, 2006, 73:1223-9). At least 5% of adults 85 years or older have DLB, which is characterized by the presence of LB in the subcortical and cortical (frontotemporal) regions of the brain. LB are composed of abnormal aggregations of α-Syn causing neurodegeneration. The clinical features of DLB include dementia (executive function deficit, visuospatial impairment), delirium, visual hallucinations, parkinsonism (bradykinesia, rigidity, tremors), and depression. Like PD, therapies to treat symptoms include regulation of dopamine levels to improve mobility of DLB patients and administration of cholinesterase inhibitors to treat delirium and visual hallucination symptoms.

PD and DLB present a huge strain on the health care system financially due to therapeutic expenditures for home care and hospitalization. Individuals affected by these diseases often require constant care towards the end of their lives. A variety of therapies to treat the symptoms of PD and DLB are available today, however decreased levels of α-Syn which comprise the Lewy bodies (disease modifying therapy) is thought to be the most effective means for disease management and eradication.

### BRIEF SUMMARY OF THE INVENTION

The present invention includes methods of treating Parkinson's disease (PD), dementia with Lewy Bodies, and associated conditions, comprising administering a therapeutically effective dose of tissue kallikrein (KLKI), variants, or active fragments thereof.

In some embodiments, the associated conditions can be Parkinson's plus syndromes or synucleinopathies, or a combination thereof.

In another aspect, the invention includes a method of improving cleavage of α-synuclein fibrils by administering KLK1, or a variant or an active fragment thereof.

In another aspect, the invention includes a method the breakdown of α-synuclein fibrils by administering KLK1, or a variant or an active fragment thereof.

In another aspect, the invention includes a method of improving neurovasculature by administering KLK1 or a variant or an active fragment thereof.

In another aspect, the invention includes a method of improving oxygen uptake to the brain by administering KLK1, or a variant or an active fragment thereof.

In another aspect, the invention includes a method improving blood flow to the brain by administering KLK1, or a variant or an active fragment thereof.

In another aspect, the present invention includes improved glucose uptake by the brain by administering KLK1, or a variant or an active fragments thereof.

In another aspect, the invention includes a method of improving dopaminergic levels in the brain by administering KLK1, or a variant or an active fragment thereof.

In another aspect of the present invention, tissue kallikrein, or a variant or an active fragment thereof, can be administered orally. Oral administration may be an enteral administration, such as a liquid, pill, or capsule to be swallowed.

In another aspect of the present invention, tissue kallikrein, or a variant or an active fragment thereof, can be administered intranasally.

In a further aspect of the present invention, an oral therapeutic dose can be a maximum dose range of about 0.001 to about 1000 International Units (IU) per day.

In a further aspect of the present invention, a nasal therapeutic dose is a maximum dose of about 0.001 to about 5000 IU per day.

Another aspect of the present invention includes a method comprising administering 1) KLK1, or a variant or an active fragment thereof, and 2) an additional therapeutic compound useful in treating PD. A PD therapeutic compound includes, but is not limited to, an anticholinergic agent, an antiinfective agent, a catechol-O-methyl (COMT) transferase, a dopamine agonist, a monoamine oxidase type B (MAO-B) inhibitor, a neurological agent, a nutritional supplement, a psychotrophic agent, or an antidepressant, or a combination thereof.

A further aspect of the invention wherein an anticholinergic agent that can be benztropine, orphenadrine, procyclidine, or trihexyphenidyl, or a combination thereof.

A further aspect of the invention wherein an antiinfective agent that can be amantadine.

A further aspect of the invention wherein a catechol-O-methyl (COMT) transferase can be carbidopa, entacapone, levodopa or tolcapone, or a combination thereof.

A further aspect of the invention includes a dopamine agonist that can be apomorphine, bromocriptine, cabergoline, pergolide, pramipexole, or ropinirole, or a combination thereof.

A further aspect of the invention includes a monoamine oxidase type B (MAO-B) inhibitor that can be, rasagiline or selegiline, or a combination thereof.

A further aspect of the invention includes a neurological agent that can be brasofensine (investigational), istradefylline (investigational) or leteprinim, or a combination thereof.

A further aspect of the invention includes a nutritional supplement that can be co-enzyme Q-10 and ubiquinone or creatine, or a combination thereof.

A further aspect of the invention includes a psychotrophic agent that can be diphenhydramine.

A further aspect of the invention includes an antidepressant that can be a selective serotonin reuptake inhibitor (SSRI), a tricyclic antidepressant, or another antidepressant drug, or a combination thereof.

A further aspect of the invention includes a selective serotonin reuptake inhibitor (SSRI) that can be citalopram, fluoxetine, paroxetine, or sertaline, or a combination thereof.

A further aspect of the invention includes a tricyclic antidepressant that can be amitriptyline, imipramine, lofepramine, or nortriptyline, or a combination thereof.

A further aspect of the invention includes another antidepressant drug that can be mirtrazapine, moclobemide, phenelzine, or venlafaxine, or a combination thereof.

Another embodiment of the present invention includes a method comprising deep brain stimulation device and administering tissue kallikrein, or a variant or active fragment thereof.

In a further embodiment of the invention, a method of treating PD or DLB may be assessed and monitored by the use of standardized scales.

In a further embodiment of the invention, standardized scales include, but are not limited to, the Modified Hoehn & Yahr Scale, Unified Parkinson's Disease Rating Scale (UPDRS), Schwab and England Scale, or Parkinson's Disease Questionnaire (PDQ 39), or a combination thereof.

Another embodiment of the present invention includes a composition formulated for oral administration comprising about 0.001 to about 1000 IU of KLKI, or a variant or an active fragment thereof, optionally further comprising a pharmaceutically acceptable excipient, and optionally further comprising an additional therapeutic compound as described above.

Another embodiment of the present invention includes a composition formulated for intranasal administration comprising about 0.001 to about 5000 IU of KLKI, or a variant or an active fragment thereof, optionally comprising a pharmaceutically acceptable excipient.

In further embodiments of the invention, a therapeutically effective amount of tissue kallikrein, or a variant or active fragment thereof is administered intranasally.

In further embodiments of the invention, a therapeutically effective dose is about 0.001 to about 5000 International Units (IU) dosage frequency.

In further embodiments of the invention, a therapeutically effective amount of tissue kallikrein, or a variant or active fragment thereof is administered orally.

In further embodiments of the invention, a therapeutically effective of tissue kallikrein, or a variant or active fragment thereof is about 0.001 to about 1000 IU per day.

### BRIEF DESCRIPTION OF THE FIGURES

FIG. 1 shows that treatment with KLK1 leads to significant degradation of α-synuclein. After 24 hour incubation at 37° C., samples were assayed by western blot and stained with N-terminal α-synuclein antibody (ab21975). Lane 1 is 100 nM KLK1+2.5 μM α-synuclein, lane 2 is 100 nM KLK1 alone, lane 3 is 2.5 μM α-synuclein alone, and lane 4 is protein marker standard (kDa).

FIG. 2 shows that treatment with KLK1 leads to significant degradation of α-synuclein. After 24 hour incubation at 37° C., samples were assayed by western blot and stained with C-terminal α-synuclein antibody (ab6162). Lane 1 is 100 nM KLK1+2.5 μM α-synuclein, lane 2 is 100 nM KLK1 alone, lane 3 is 2.5 μM α-synuclein alone, and lane 4 is protein marker standard (kDa).

FIG. 3 shows that treatment with KLK1 leads to significant degradation of α-synuclein 1-95. After 3 hours of incubation at 37° C., samples were assayed by western blot with α-synuclein NAC domain antibody (5C2). Lane 1 is 1 mM KLK1 alone, lane 2 is 250 μg/ml α-synuclein 1-95 alone, lane 3 is 1 mM KLK1 and 250 μg/ml α-synuclein 1-95, and lane 4 is protein marker standard (kDa).

FIG. 4 shows a Western blot of recombinant human KLK1. Various amounts of purified recombinant human KLK1 were electrophoresed on a SDS-PAGE gel and then stained with Coomassie Blue stain. Lane 1 is a pre-stained protein ladder, the molecular weights of the standards are written on the side (in kDa). Lanes 2-5 have KLK1 purified from CHO cells (lane 2, 14 μg protein; lane 3, 7 μg protein; lane 4, 3.5 μg protein; lane 5, 1.35 μg protein). Lane 6 has 14 μl of KLK1 protein purified from transient transfection of 293 cells.

FIG. 5 shows a Western blot of various amounts of recombinant human KLK1 purified from CHO or 293 cell lines following transient transfection and stained with mouse anti-human KLK1 polyclonal antibodies. Lanes 1 and 6 are loaded with a pre-stained protein ladder, the molecular weights of the standards are written on the side (in kDa). Lanes 2-5 have KLK1 purified from CHO cells (lane 2, 5 μl protein; lane 3, 2.5 μl protein; lane 4, 1.25 μl protein). Lane 5 has 2.5 μl of KLK1 protein purified from transient transfection of 293 cells.

FIG. 6 shows that recombinant human KLK1 promotes general neuronal cell protection in nigral ventral mesencephalic cell culture after serum withdrawal (PCM_{—}0% FBS). Cell cultures were graded on a general neuronal cell culture scale (1-10, 0 being no sign of viable neuronal cells and 10 being no sign of cell death) on the y-axis at 4 hours post plating, and on DIV 1 (Day in vitro 1), DIV 2 and DIV 3. The various culture conditions were (on the x-axis) 1: Negative control (PCN-0), 2: Positive control (VMCL1-25), 3: Positive control (PCM-5), 4: Human recombinant KLK1 (10 U/ml), 5: Human recombinant KLK1 (5 U/ml), 6: Human recombinant KLK1 (1 U/ml), 7: Human recombinant KLK1 (0.2 U/ml), and 8: Human recombinant KLK1 (0.04 U/ml).

FIG. 7 shows that recombinant human KLK1 promotes a dose dependent protection of dopaminergic neurons (TH+) and general neuronal population (MAP2+) after serum withdrawal (PCM_{—}0% FBS). The nigral cell cultures were incubated in various culture medium as depicted on the x-axis; 1: Negative control (PCN-0), 2: Positive control (VMCL1-25), 3: Positive control (PCM-5), 4: Human recombinant KLK1 (10 U/ml), 5: Human recombinant KLK1 (5 U/ml), 6: Human recombinant KLK1 (1 U/ml), 7: Human recombinant KLK1 (0.2 U/ml), and 8: Human recombinant KLK1 (0.04 U/ml). On DIV-3, the cell cultures were immunohistologically stained with antibodies TH (top graph) and MAP2 antibodies (lower graph), and the average number of positive neurons per field+/−SD are depicted on the graph.

FIG. 8 shows that recombinant human KLK1 protects dopaminergic neuronal cells in culture from the neurotoxic action of 6-hydroxydopamine (6-OHDA). The nigral cell cultures were incubated in various culture medium as depicted on the x-axis; 1: Negative control (PCN-0), 2: Positive control (PCM-2.5), 3: Positive control (PCM-5), 4: Human recombinant KLK1 (10 U/ml), 5: Human recombinant KLK1 (5 U/ml), 6: 6-OHDA (30 uM), 7: Human recombinant KLK1 (10 U/ml)+6-OHDA (30 uM), and 8: Human recombinant KLK1 (5 U/ml)+6-OHDA (30 uM). On DIV-3, the cell cultures were immunohistologically stained with antibodies to TH (top graph) and MAP2 (lower graph), and the average number of positive neurons per field+/−SD are depicted in the graph.

### DETAILED DESCRIPTION

### Definitions

“Tissue kallikrein” or “KLK1” is a serine protease that is primarily noted for its role in controlling hypertension through its cleavage of kininogen into lysyl-bradykinin (kallidin) (Yousef et al., Endocrine Rev. 2001; 22: 184-204). As there are a large number of enzymes in the KLK family, the inventors believe that KLK1 appears to be a ubiquitous or multiple target acting enzyme, in addition to its recognized role in hypertension regulation and as such may specifically play an important role in treating Parkinson's disease. As used herein, the term “tissue kallikrein” is synonymous with the following terms: callicrein, glumorin, padreatin, padutin, kallidinogenase, bradykininogenase, pancreatic kallikrein, onokrein P, dilminal D, depot-Padutin, urokallikrein, or urinary kallikrein.

In some aspects, tissue kallikrein polypeptide can have the following sequence:

NP_001001911 GI: 50054435 Sus scrofa
1-17 signal peptide
18-24 propeptide
25-263 mature peptide
>gi|50054435|ref/NP_001001911.1| kallikrein 1
[Sus scrofa]
(SEQ ID NO: 1)
MWSLVMRLALSLAGTGAAPPIQSRIIGGRECEKDSHPWQVAIYHYSSFQC
GGVLVDPKWVLTAAHCKNDNYQVWLGRHNLFENEVTAQFFGVTADFPHPG
FNLSLLKNHTKADGKDYSHDLMLLRLQSPAKITDAVKVLELPTQEPELGS
TCQASGWGSIEPGPDDFEFPDEIQCVELTLLQNTFCADAHPDKVTESMLC
AGYLPGGKDTCMGDSGGPLICNGMWQGITSWGHTPCGSANKPSIYTKLIF
YLDWINDTITENP

Another embodiment includes a human tissue kallikrein polypeptide that has the following sequence:

NP_002248 GI: 4504875 Homo sapiens
1-18 signal peptide
19-24 propeptide
25-262 mature peptide
>gi|4504875|ref|NP_002248.1| kallikrein 1
preproprotein [Homo sapiens]
(SEQ ID NO: 2)
MWFLVLCLALSLGGTGAAPPIQSRIVGGWECEQHSQPWQAALYHFSTFQC
GGILVHRQWVLTAAHCISDNYQLWLGRHNLFDDENTAQFVHVSESFPHPG
FNMSLLENHTRQADEDYSHDLMLLRLTEPADTITDAVKVVELPTEEPEVG
STCLASGWGSIEPENFSFPDDLQCVDLKILPNDECKKAHVQKVTDFMLCV
GHLEGGKDTCVGDSGGPLMCDGVLQGVTSWGYVPCGTPNKPSVAVRVLSY
VKWIEDTIAENS

When comparing the Genbank amino acid sequence for human KLK1 to the amino acid sequence of a cDNA for human KLK1 purchased from Origene (Rockville, Md., USA), two apparent amino acid substitutions were detected that may result from single-nucleotide polymorphism or SNP's between individuals within a species. The SNP's result in an apparent E to Q at amino acid residue 145 of 262, and an apparent A to V position 188 of 262, as depicted in SEQ ID NO:3. The inventors proceeded with expressing, purifying and testing the pre-pro-human KLK1 polypeptide described in SEQ ID NO:3.

1-18 signal peptide
19-24 propeptide
25-262 mature peptide
(SEQ ID NO: 3)
MWFLVLCLALSLGGTGAAPPIQSRIVGGWECEQHSQPWQAALYHFSTFQC
GGILVHRQWVLTAAHCISDNYQLWLGRHNLFDDENTAQFVHVSESFPHPG
FNMSLLENHTRQADEDYSHDLMLLRLTEPADTITDAVKVVELPTQEPEVG
STCLASGWGSIEPENFSFPDDLQCVDLKILPNDECKKVHVQKVTDFMLCV
GHLEGGKDTCVGDSGGPLMCDGVLQGVTSWGYVPCGTPNKPSVAVRVLSY
VKWIEDTIAENS

In some aspects, a human tissue kallikrein gene of the present invention encodes a 262-amino acid tissue kallikrein polypeptide: a presumptive 17-amino acid signal peptide, a 7-amino acid proenzyme fragment and a 238-amino acid mature KLK1 protein. Hence, certain KLK1 polypeptides comprise or consist of residues 1-263, 18-263, or 25-263 of SEQ ID NO:1, or residues 1-262, 19-262, or 25-262 of SEQ ID NO: 2, or 3.

The term “active fragment” refers to smaller portions of a KLK1 polypeptide that retain the serine protease activity of a full-length KLK1 polypeptide.

A “variant” or “mutant” of a starting or reference polypeptide is a polypeptide that 1) has an amino acid sequence different from that of the starting or reference polypeptide and 2) was derived from the starting or reference polypeptide through either natural or artificial (manmade) mutagenesis. Such variants include, for example, deletions from, and/or insertions into and/or substitutions of, residues within the amino acid sequence of the polypeptide of interest. A variant amino acid, in this context, refers to an amino acid different from the amino acid at the corresponding position in a starting or reference polypeptide sequence. Any combination of deletion, insertion, and substitution may be made to arrive at the final variant or mutant construct, provided that the final construct possesses the desired functional characteristics. Amino acid changes may also alter post-translational processes of the polypeptide, such as changing the number or position of glycosylation sites. Methods for generating amino acid sequence variants of polypeptides are described in U.S. Pat. No. 5,534,615, expressly incorporated herein by reference.

A “wild type” or “reference” sequence or the sequence of a “wild type” or “reference” protein/polypeptide maybe the sequence from which variant polypeptides are derived through the introduction of mutations. In general, the “wild type” sequence for a given protein is the sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

“Percent (%) amino acid sequence identity” with respect to the polypeptides identified herein is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. The ALIGN-2 program is publicly available through Genentech, Inc., South San Francisco, Calif.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y,

where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Percent (%) nucleic acid sequence identity” is defined as the percentage of nucleotides in a candidate sequence that are identical with the nucleotides in a reference polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z,

where W is the number of nucleotides scored as identical matches by the sequence alignment program in that program's alignment of C and D, and where Z is the total number of nucleotides in D. It will he appreciated that where the length of nucleic acid sequence C is not equal to the length of nucleic acid sequence D, the % nucleic acid sequence identity of C to D will not equal the nucleic acid sequence identity of D to C.

The term “amino acid” is used in its broadest sense and is meant to include the naturally occurring L α-amino acids or residues. The commonly used one and three letter abbreviations for naturally occurring amino acids are used herein (Lehninger, A. L., Biochemistry, 2d ed., pp. 71-92, (1975), Worth Publishers, New York). The term includes all D-amino acids as well as chemically modified amino acids such as amino acid analogs, naturally occurring amino acids that are not usually incorporated into proteins such as Norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio, In: The Peptides: Analysis, Synthesis, Biology, Gross and Meiehofer, Eds., Vol. 5 p 341, Academic Press, Inc, N.Y. 1983, which is incorporated herein by reference.

The term “protein” has an amino acid sequence that is longer than a peptide. A “peptide” contains 2 to about 50 amino acid residues. The term “polypeptide” includes proteins and peptides. Examples of proteins include, but are not limited to, antibodies, enzymes, lectins and receptors; lipoproteins and lipopolypeptides; and glycoproteins and glycopolypeptides.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the two portions of the polypeptide are obtained from heterologous or different polypeptides.

“α-synuclein (αsyn or α-Syn)” refers to a 140 amino acid protein that is found in elevated concentrations in the neocortex, hippocampus and substantia nigra, mainly at the synapse, all of which are seen to be altered in the preliminary stages of PD (Suh et al., 2002). Localization of α-Syn suggests a role in the regulation of dopamine release, storage, uptake, and synthesis. α-Syn is known to readily undergo post-translational phosphorylation and can be observed at such elevated levels of phosphorylation in PD and DLB (Suh et al., 2002). Misfolding, oligomerization, and fibrillization of α-Syn are believed to be involved in PD and related disorders progression. α-Syn is highly susceptible to aggregation due to its hydrophobic amino acid portion (Suh et al., 2002), resulting in the formation of intracytoplasmic Lewy bodies. Lewy Bodies have been noted to be enriched with α-Syn, lacking their C-terminus, a form which is noted for its greater rate of aggregation as the C-terminal protection of the aggregation region of α-Syn is absent. Additionally, mutations within the α-Syn protein sequence (e.g., A30P, A53T) also lead to increased aggregation of α-Syn. The formation of Lewy bodies via α-Syn aggregation leads to the generation of reactive oxidative species, a key factor responsible for the death of dopaminergic neurons in the SNc (Junn, et al, Neurosci Lett. 2002, 320; 146-150). α-Syn is also able to interact with proteins of the Erk cascade and protein kinase C, which may be involved in a mechanism of neuronal cell death (Suh, 2002). The toxicity of α-Syn beyond the intracytoplasmic space of neurons, has been shown with extracellular forms of α-Syn (fibril and oligomer) being secreted by neurons (Lee, 2008). Extracellular α-Syn may exert its toxic effects through either inserting into the plasma membrane of neurons or activating microglial cells which generate free radicals. α-Syn's oligomeric intermediate form is thought to be the most toxic α-Syn species (Tetzlaff et al., J. Biol. Chem., 2008, 283:17962-17968).

The term “Parkinson-plus syndromes”, as used herein, refers to a group of conditions manifested by the classical features of PD (known as parkinsonism which includes tremor, bradykinesia and muscular rigidity) with additional features which distinguish them from simple idiopathic PD. These conditions include, but are not limited to, Multiple System Atrophy (MSA), Progressive Supranuclear Palsy (PSP) or Corticobasal Degeneration (CBD). These conditions, among others, may also be grouped together as “synucleinopathies” as used herein, which refers to conditions which share common pathological aggregates of α-Syn in selected population of neuronal or glial cells of the brain.

The term “therapeutically effective amount” refers to an amount of a composition of this invention effective to “alleviate” or “treat” a disease or disorder in a subject or mammal. Generally, alleviation or treatment of a disease or disorder involves the lessening of one or more symptoms or medical problems associated with the disease or disorder. In some embodiments, it is an amount that improves neurovasculature, oxygen uptake, blood flow, glucose uptake, dopaminergic levels, or cleavage of fibrils, breakdown of fibrils or a combination thereof.

The terms “treatment” and “treating” refer to inhibiting, alleviating, and healing Parkinson's disease, conditions or symptoms thereof. “Treating” or “treatment” refers to both therapeutic treatment and prophylactic or preventative measures, wherein the object is to prevent or slow down (lessen) the targeted pathologic condition or disorder. Treatment can be carried out by administering a therapeutically effective amount of at least one compound of the invention, and assessed using standardized scales as described herein.

The term “improving neurovasculature” refers to an increase of blood vessel density or increased nutrient delivery to the brain through the blood vessel network. Use of high resolution magnetic resonance imaging (MRI) allows for development of a three dimensional (3D) vascular network map of an imaged brain. Use of endogenous blood oxygenation level-dependent contrast and exogenous contrast agent allows for visualization of artery and vein structures within a 3D image (Bolan et. al, 2006). Comparison of a vascular network before treatment, during treatment, and after treatment allows for assessment of improved neurovasculature for a particular PD or DLB patient undergoing treatment. An “increase” refers to a greater blood vessel density or greater nutrient delivery to the brain in a patient after treatment compared to a blood vessel density or nutrient delivery in the patient before treatment.

The term “improving oxygen uptake” refers to the increased delivery of oxygen to the brain and cells of the brain while the term “improving blood flow” refers to an increase of blood volume circulating through the brain. Use of functional MRI allows for visualization of blood flow in the brain (Davis et. al, 1998). An area of brain that undergoes activity requires oxygen to aid in the metabolism of glucose for energy. This is achieved by a large increase in blood flow so that a diffusion limitation of oxygen is overcome and is supplied in plentiful amounts to active brain tissue. This increase in blood flow and accompanying increase in oxygen is detected through changes in endogenous blood oxygenation level-dependent contrast by functional MRI (Nandhagopal, et al. Neurology 2008, 70:1478-1488). Increased signal is then used to derive the increase in blood flow, oxygen uptake, and metabolism. By mapping areas of blood flow and oxygen uptake deficiencies in the brain of a PD or DLB patient, improvement can be assessed during and after treatment using age matched non-PD or non-DLB patient as a control. An “increase” refers to greater oxygen uptake or blood flow in a patient after treatment compared to oxygen uptake or blood flow in the patient before treatment.

The term “improved glucose uptake” refers to an enhanced ability of the brain to utilize glucose from the blood stream. In PD and DLB there is a reduction of glucose uptake and metabolism by cells of the brain (hypometabolism); this marker of disease onset is determined by the use of PET imaging of the brain with fluorine labeled glucose contrast agent (FDG-PET). By comparing images generated by this method before, during, and after treatment, an improvement in glucose uptake in areas of the brain in a PD or DLB patient previously displaying a reduction of glucose uptake can be assessed while using age-matched non-PD or DLB control subjects.

The term “improving dopaminergic levels” refers to an enhanced ability of the brain to take up L-3,4-dihydroxyphenylalanine (L-DOPA) and intracellular conversion into dopamine which translates into an increase of dopaminergic neurons in the brain. Uptake of L-DOPA is determined by the use of PET imaging of the brain with fluorine labeled 6-F¹⁸-L-DOPA contrast agent (FD PET) (Nandhagopal, et al. 2008). In early stages of Parkinson's Disease, uptake of L-DOPA may increase to compensate for loss of dopaminergic neurons by an increase of activity L-DOPA conversion to dopamine by remaining dopaminergic neurons. This compensatory mechanism disappears as Parkinson's disease progresses leading to a marked drop in L-DOPA uptake measurements by PET scan. By comparing images generated by this method before, during, and after treatment, an improvement in dopaminergic levels in areas of the brain in a PD or DLB patient previously displaying a reduction of dopaminergic levels can be assessed while using age-matched non-PD or non-DLB control subjects.

As well, for DLB, levels of dopaminergic neurons can be assessed by the level of dopamine transporter detection. Dopamine transporter mediates the re-uptake of dopamine from the synaptic cleft and its level is determined by SPECT imaging of the brain with iodine I¹²³-radiolabeled 2β-carbomethoxy-3β-(4-iodophenyl)-N-(3-fluoropropyl) nortropane (FP-CIT) contrast, agent which has affinity to the transporter.

By comparing images generated by this method before, during, and after treatment, an improvement in dopaminergic levels in areas of the brain in a DLB patient previously displaying a reduction of dopaminergic levels can be assessed while using age-matched non-DLB control subjects.

The term “improving cleavage of α-synuclein fibrils” refers to the enhanced ability to proteolytically digest α-synuclein fibrils.

The term “breakdown of α-synuclein fibrils” refers to the outcome of the proteolytic cleavage of α-synuclein fibrils.

The term “standardized scales”, as used herein, refers to questionnaires and inventories, which may be used to assess an individual with Parkinson's disease or Dementia with Lewy Bodies, and can include, but are not limited to, the Modified Hoehn & Yahr Scale, Unified Parkinson's Disease Rating Scale (UPDRS), Schwab and England Scale, or Parkinson's Disease Questionnaire (PDQ 39).

The term “Modified Hoehn & Yahr Scale”, as used herein, refers to, a scale used to assess the degree of disease progression characterized by a numerical value, Stage 0-V where Stage 0 indicates complete absence of the disease while Stage V indicates the disease in its most progressed form. This scale can be seen in Recent Developments in Parkinson's Disease (Fahn S, Marsden C D, Calne D B, Goldstein M, eds. Recent Developments in Parkinson's Disease, Vol 2. Florham Park, N.J. Macmillan Health Care Information 1987, pp 15 3-163, 293-304) and is available online at http://www.mdvu.org/library/ratingscales/pd/updrs.pdf, accessed Jul. 23, 2008.

The term “Unified Parkinson's Disease Rating Scale” or “UPDRS”, as used herein, refers to a scale used to assess the degree of disease progression by evaluation from a physician as well as patient self evaluation resulting in a point score of 0-199, where 0 indicates complete absence of the disease while 199 indicates the disease in its most progressed form. This scale can be seen in Recent Developments in Parkinson's Disease (Fahn S, Marsden C D, Calne D B, Goldstein M, eds. Recent Developments in Parkinson's Disease, Vol 2. Florham Park, N.J. Macmillan Health Care Information 1987, pp 15 3-163, 293-304) and is available online at http://www.mdvu.org/library/ratingscales/pd/updrs.pdf, accessed Jul. 23, 2008.

The term “Schwab and England Scale” as used herein, refers to, a scale which quantifies a patients ability to perform tasks and their level of independence upon task performance and completion as an indication of disease progression. This scale can be seen in Recent Developments in Parkinson's Disease (Fahn S, Marsden C D, Calne D B, Goldstein M, eds. Recent Developments in Parkinson's Disease, Vol 2. Florham Park, N.J. Macmillan Health Care Information 1987, pp 15 3-163, 293-304) and is available online at http://www.mdvu.org/library/ratingscales/pd/updrs.pdf, accessed Jul. 23, 2008.

The term “Parkinson's Disease Questionnaire” or “PDQ 39”, as used herein, refers to a self-administered questionnaire provided to patients to determine the degree of disease progression where a low score indicates the disease at a mild stage while a higher score indicates a more progressed stage of the disease, as described by Katsarou (Katsarou et al., Quality of Life Research, 2001, 10: 159-163).

Methods for Generating cDNA Coding for Recombinant Human KLK1 and Expression Vectors.

According to the present invention, DNA sequences encoding a human KLK1 polypeptide of the present invention have been isolated and characterized. Further, human DNA sequences may be utilized in eukaryotic and prokaryotic expression systems to provide isolatable quantities of recombinant KLK1 protein having one or more of the biological and immunological properties of naturally-occurring KLK1, including one or more in vivo and/or in vitro biological activities (e.g., therapeutic activities) of naturally-occurring KLK1.

Prokaryotic or eukaryotic host expression (e.g., by bacterial, yeast and mammalian cells in culture) of exogenous DNA of the present invention obtained by genomic or cDNA cloning or by gene synthesis yields recombinant human KLK1 polypeptides described herein. KLK1 polypeptide products of cell culture expression in vertebrate (e.g., mammalian and avian) cells may be further characterized by freedom from association with human proteins or other contaminants, which may be associated with KLK1 in its natural mammalian cellular environment or in extracellular fluids such as plasma or urine. Products of typical yeast (e.g., Saccharomyces cerevisiae) or prokaryote (e.g., E. coli) host cells are free of association with any mammalian proteins. Depending upon the host employed, polypeptides of the invention may be glycosylated with mammalian or other eukaryotic carbohydrates or may be non-glycosylated. Polypeptides of the invention may also include an initial methionine amino acid residue (at position-1).

Illustrative of the present invention are cloned DNA sequences of human species origins and polypeptides suitably deduced therefrom which represent, respectively, the primary structural conformation of KLK1 of human species origins.

The cell culture expressed KLK1 polypeptides of the present invention may be isolated and purified by conventional means including, e.g., chromatographic separations or immunological separations involving monoclonal and/or polyclonal antibody preparations, or using inhibitors or substrates of serine proteases for affinity chromatography. Polypeptide products of the invention may be “labeled” by covalent association with a detectable marker substance (e.g., radiolabels such as I¹²⁵ or P³², nonisotopic labels such as biotin) to provide reagents useful in detection and quantification of KLK1 in solid tissue and fluid samples such as blood or urine. DNA products of the invention may also be labeled with detectable markers (e.g., radiolabels such as I¹²⁵ or P³², nonisotopic labels such as biotin) and employed in DNA hybridization processes to locate the KLK1 gene position and/or the position of any related gene family in a human, monkey and other mammalian species chromosomal map. The labeled DNA may also be used for identifying the KLK1 gene disorders at the DNA level and used as gene markers for identifying neighboring genes and their disorders.

Tissue kallikrein polypeptide products provided by the invention are products having a primary structural conformation of a naturally-occurring tissue kallikrein to allow possession of one or more of the biological properties thereof and having an average carbohydrate composition which may differ from that of naturally-occurring tissue kallikrein.

KLK1 is a serine protease which cleaves low-molecular-weight kininogen resulting in the release of kallidin (lys-bradykinin). This activity of KLK1 may be measured in an enzyme activity assay by measuring either the cleavage of low-molecular-weight kininogen, or the generation of lys-bradykinin. Assays include examples wherein a labeled substrate is reacted with KLK1, and the release of a labeled fragment may be detected. One example of such a fluorogenic substrate suitable for KLK1 measurement of activity is D-val-leu-arg-7 amido-4-trifluoromethylcoumarin (D-VLR-AFC, FW 597.6) (Sigma, Cat #V2888 or Ana Spec Inc Cat #24137.) When D-VLR-AFC is hydrolyzed, the free AFC produced in the reaction can be quantified by fluorometric detection (excitation 400 nm, emission 505 nm) or by spectrophotometric detection at 380 nm (extinction coefficient=12,600 at pH 7.2). Other methods and substrates may also be used to measure KLK1 proteolytic activity.

KLK1 activity, measured in Units or Units/ml, may be determined by comparing the relative activity of a KLK1 sample to the Kininogenase, Porcine standard acquired from the National Institute for Biological Standards and Control (NIBSC Product No. 78/543). For this standard, the assigned potency is 22.5 international units (IU) per 20 μg ampoule of porcine pancreatic kininogenase. Typically, serial dilutions are made of the standard, and the activity in an unknown sample of KLK1 is compared to the standard.

Methods of Treating Parkinson's Disease and Dementia with Lewy Bodies.

Treatment of PD symptoms primarily focuses on regulating the levels of dopamine by increasing dopamine levels (e.g., levodopa given with carbidopa), the use of dopamine agonists (e.g., Bromocriptine), or by decreasing the level of acetylcholine (e.g., anticholinergics) to compensate for reduced dopamine levels. These therapies along with physiotherapy are used to help improve mobility. Treatment of depression symptoms in those with PD can be managed with tricyclic antidepressants, selective serotonin re-uptake inhibitors, or monoamine oxidase A inhibitors. However, a number of antidepressants, namely D2 antagonist based typical antipsychotics (neuroleptics), should be avoided, as PD symptoms can be made worse.

The present invention provides methods for treating Parkinson's disease, dementia with Lewy Bodies, and conditions associated therewith. One embodiment includes a method of treating Parkinson's disease, and/or dementia with Lewy Bodies by administering a therapeutically effective amount of tissue kallikrein, or variant or active fragment thereof. Tissue kallikrein, or variant or active fragment thereof, can be administered to a mammal, preferably, a human.

Activation of MMP9 from its inactive pro-MMP9 form is thought to occur by the action of extracellular proteases, including KLK1 (Desrivieres et. al, J Cell Physiol, 1993, 157(3): 587-93). Studies have shown the ability of MMP9 to cleave α-Syn at four sites in the NAC region, thereby resulting in depleted α-Syn aggregation and complete breakdown of α-Syn (Sung et al., The Journal of Biological Chemistry, 2005, 280(26):25216-25224). The protease activity of KLK1 is likely responsible for the cleavage of α-Syn either directly or indirectly (by MMP-9 activation).

The 1-methyl-4-phenyl-1,2,3,6-tetrahydropyridine (MPTP) mouse model of PD is directly linked to the upregulation of α-Syn expression and increased levels of hyperphosphorylated Tau protein. MPTP induction of PD results in an abrupt activation of GSK-3β, which is known to play a role in the neurotoxic action of MPTP, resulting in apoptosis and therefore may be a target for disease treatment (Wang et al., Neuro Pharmacology, 2007, 52:1678-1684). Inhibition of GSK-1β protected against MPTP mediated cell death of dopamingeric neurons and resulted in an improvement in animal behavior.

The activity of GSK-3β is normally regulated by serine 9 phosphorylation by Akt. Activation of the kinin B₂ receptor signaling pathway by kinin (Yin et al., J Biol Chem, 2005, 280(9): 8022-30) and the NGF-acetylcholine pathway leads to increased GSK-3β phosphorylation (Rylett et al., J Neurosci, 1993, 13(9): 3956-3963) (De Sarno et al., Neurobiol Aging, 2006, 27(3): 413-22.). Both pathways are thought to be mediated by extracellular proteases, including KLK1 (Castro et al., FEBS Lett, 1990, 267(2): 207-12) (Xia et al., Hypertension, 2006, 47(4): 752-61).

Additional routes of activation of the PI3K/Akt/GSK-3β pathway can be achieved by the binding of neurotrophins, such as NGF, to their respective Trk receptors, (Trk A for NGF). Neurotrophins are noted for their ability to provide neuroprotection from apoptosis and neurodegeneration, promote axonal growth and improve neuronal synaptic connectivity (Buckley et al., Schizophr Res, 2007, 94(1-3): 1-11). As well, binding to their appropriate Trk receptor leads to an increase in their production. The binding of NGF to Trk A, leading to Akt activation, can only take place once NGF has been cleaved into its mature form. The precursor form of NGF is post-transitionally modified by KLK1 cleavage into its mature form such that NGF is able to activate the PI3K/Akt/GSK-3β pathway through Trk A. Animal models and patients with PD have shown reduced NGF levels (Lorigados, et al., Brain Res. 2002 952:122-127) and that signaling through NGF can protect from MPTP mediated cell death (Shimoke, et al., J Neurosci Res. 2001 63:402-409).

As such, a method of treating PD or DLB through the administration of KLK1, a variant or active fragment thereof, either orally or via the intranasal route, improves GSK-3β regulation in the brain.

### Administration of Tissue Kallikrein

Traditional modes of drug administration to treat aliments in the brain include oral as well as intravenous routes of administration. These modes are not always ideal. Oral administration of compounds results in limited bioavailability (solubility, 1^{st} pass liver degradation, blood brain barrier restriction) as well as time release issues with potentially undesirable gastrointestinal side effects. However, Tissue Kallikrein (KLK1) appears able to pass through and bypass the blood-brain-barrier such that it may produce its effects on the brain.

An oral dose of KLKI, variant, or active fragment thereof, can be a dose of about 1 to about 1000 IU per day; about 1 to about 750 IU per day; about 1 to about 500 IU per day; about 1 to about 400 IU per day; about 1 to about 300 IU per day; about 1 to about 250 IU per day; about 1 to about 200 IU per day; about 1 to about 150 IU per day; about 1 to about 100 IU per day; about 1 to about 75 IU per day; about 1 to about 50 IU per day; about 1 to about 50 IU per day; about 1 to about 25 IU per day; about 1 to about 20 IU per day; about 1 to about 15 IU per day; about 1 to about 10 IU per day; about 1 to about 5 IU per day; about 5 to about 1000 IU per day; about 10 to about 1000 IU per day; about 15 to about 1000 IU per day; about 20 to about 1000 IU per day; about 25 to about 1000 IU per day; about 50 to about 1000 IU per day; about 75 to about 1000 IU per day; about 100 to about 1000 IU per day; about 150 to about 1000 IU per day; about 200 to about 1000 IU per day; about 250 to about 1000 IU per day; about 300 to about 1000 IU per day; about 400 to about 1000 IU per day; about 500 to about 1000 IU per day; about 750 to about 1000 IU per day; about 10 to about 100 IU per day; about 10 to about 250 IU per day; about 10 to about 500 IU per day; about 50 to about 250 IU per day; about 50 to about 500 IU per day; about 100 to about 250 IU per day; about 100 to about 500 IU per day; or about 250 to about 750 IU per day.

Intravenous (i.v.) administration may require trained medical professionals, which is time consuming and costly to the health care system and may result in patient compliance issues. Risks associated with intravenous administration are also present, namely infection at the injection site and a variety of safety issues to the patient and the professional administering the dose.

Intranasal administration allows a medicament to be ‘fast acting’ since it is able to reach the brain by a more direct route. Intranasal administration is convenient and virtually eliminates issues of patient compliance as seen with intravenous administration. The cells of the olfactory epithelium are selectively permeable. Proteins such as KLK1 may be able to pass through and may bypass the blood-brain-barrier via the intranasal route, such that it may produce its effects directly on the brain, thereby minimizing peripheral effects as well. This is due to involvement of the olfactory region in the upper portion of the nasal pathway.

A intranasal dose of KLKI, variant, or active fragment thereof, can be a dose of about 1 to about 5000 IU per day; about 1 to about 4000 IU per day; about 1 to about 3000 IU per day; about 1 to about 2500 IU per day; about 1 to about 2000 IU per day; about 1 to about 1000 IU per day; about 1 to about 750 IU per day; about 1 to about 500 IU per day; about 1 to about 400 IU per day; about 1 to about 300 IU per day; about 1 to about 250 IU per day; about 1 to about 200 IU per day; about 1 to about 150 IU per day; about 1 to about 100 IU per day; about 1 to about 75 IU per day; about 1 to about 50 IU per day; about 1 to about 50 IU per day; about 1 to about 25 IU per day; about 1 to about 20 IU per day; about 1 to about 15 IU per day; about 1 to about 10 IU per day; about 1 to about 5 IU per day; about 5 to about 1000 IU per day; about 10 to about 1000 IU per day; about 15 to about 1000 IU per day; about 20 to about 1000 IU per day; about 25 to about 1000 IU per day; about 50 to about 1000 IU per day; about 75 to about 1000 IU per day; about 100 to about 1000 IU per day; about 150 to about 1000 IU per day; about 200 to about 1000 IU per day; about 250 to about 1000 IU per day; about 300 to about 1000 IU per day; about 400 to about 1000 IU per day; about 500 to about 1000 IU per day; about 750 to about 1000 IU per day; about 10 to about 100 IU per day; about 10 to about 250 IU per day; about 10 to about 500 IU per day; about 50 to about 250 IU per day; about 50 to about 500 IU per day; about 100 to about 250 IU per day; about 100 to about 500 IU per day; or about 250 to about 750 IU per day.

Intranasally administered drugs can reach tissues of the brain and spinal cord using an extracellular route through perineural channels. This intranasal administration delivers a drug to the upper third (⅓) of the nasal cavity where the drug is absorbed through nasal mucosa.

There are two possible routes that a substance administered intranasally may follow at the olfactory epithelium. These are said to be intraneuronal and extraneuronal. An intraneuronal route is by uptake of peptides into olfactory neurons where peptides may travel along axons to bypass the blood-brain-barrier. Passage through unique intercellular clefts in epithelia of the olfactory region is an extracellular route that allows peptides to diffuse into the subarachnoid space. An extracellular route is more preferable due to rapid passage time to the brain, avoidance of proteolytic degradation involved in intraneuronal pathways (Born et al., Nat. Neurosci. 2002, 5(6):514-6), and rapid eliciting of biological effects at multiple sites of the brain (Throne et al. Neuroscience, 2004, 127(2): 481-96).

Although oral delivery is possible and advantageous, an otherroute of administration is intranasal due to more direct delivery of KLK1 to desired sites of action (the brain).

Pharmaceutical compositions may be administered orally or intranasally. Formulations suitable for intranasal administration comprise 0.001 to about 5000 IU per dosage frequency of tissue kallikrein, or a variant or active fragment thereof, and can be, ointments, creams, lotions, pastes, gels, sprays, aerosols, oils and the like. Solutions or suspensions can be applied directly to the nasal cavity by conventional means, for example, with a dropper, pipette or spray. Formulations can be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

Formulations for aerosol administration, particularly to the upper respiratory tract containing the nasal cavity and olfactory region, include intranasal administration. An active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. An aerosol may conveniently also contain a surfactant such as lecithin. A dose of drug may be controlled by a metered valve. Alternatively active ingredients may be provided in a form of a dry powder, for example a powder mix of the compound in a suitable powder base such as lactose, starch, starch derivatives such as hydroxypropylmethyl cellulose and polyvinylpyrrolidine (PVP). A powder carrier will form a gel in the nasal cavity. A powder composition may be presented in unit dose form for example in capsules or cartridges of e.g., gelatine or blister packs from which the powder may be administered by means of an inhaler.

Formulations suitable for oral administration comprise 0.001 to about 1000 IU per dosage frequency of tissue kallikrein, or a variant or active fragment thereof, and can be, solution, tablets, sustained release capsules, enteric coated capsules, orally disintegrating tablets and syrups.

An “effective amount” or a “therapeutically effective amount” refers to a nontoxic but sufficient amount of drug or agent to provide a desired effect. In a combination therapy, an “effective amount” of one component of the combination is an amount of that compound that is effective to provide a desired effect when used in combination with the other components of the combination. An amount that is “effective” will vary from subject to subject, depending on the age and general condition of an individual, a particular active agent or agents, and the like. An appropriate “effective” amount in any individual case may be determined using routine experimentation.

A therapeutically effective amount of a compound of the invention for treating the above-identified diseases or symptoms thereof can be administered prior to, concurrently with, or after the onset of the disease or symptom. A compound of the invention can be administered concurrently with the onset of the disease or symptom. “Concurrent administration” and “concurrently administering” as used herein includes administering a polypeptide of the invention and another therapeutic agent in admixture, such as, for example, in a pharmaceutical composition or in solution, or separately, such as, for example, separate pharmaceutical compositions or solutions administered consecutively, simultaneously, or at different times, but not so distant in time such that the compound of the invention and the other therapeutic agent cannot interact and a lower dosage amount of the active ingredient cannot be administered.

### PD Therapeutic Compounds

Another aspect of the present invention includes a method as herein described further comprising concurrently administering an additional therapeutic compound useful in treating PD or dementia with Lewy bodies. A PD therapeutic compound includes, but is not limited to, an anticholinergic agent, an antiinfective agent, a catechol-O-methyl (COMT) transferase, a dopamine agonist, a monoamine oxidase type B (MAO-B) inhibitor, a neurological agent, a nutritional supplement, a psychotrophic agent, or certain antidepressants, such as tricyclic antidepressants, or a combination thereof.

An anticholinergic agent includes, but is not limited to benztropine, orphenadrine, procyclidine, or trihexyphenidyl, or a combination thereof.

An antiinfective agent includes, but is not limited to amantadine.

A catechol-O-methyl (COMT) transferase, includes, but is not limited to carbidopa, entacapone, levodopa or tolcapone, or a combination thereof.

A dopamine agonist, includes, but is not limited to apomorphine, bromocriptine, cabergoline, pergolide, pramipexole, or ropinirole, or a combination thereof.

A monoamine oxidase type B (MAO-B) inhibitor, includes, but is not limited to rasagiline or selegiline, or a combination thereof.

A neurological agent, includes, but is not limited to brasofensine (investigational), istradefylline (investigational) or leteprinim, or a combination thereof.

A nutritional supplement, includes, but is not limited to co-enzyme Q-10 and ubiquinone or creatine, or a combination thereof.

A psychotrophic agent, includes, but is not limited to diphenhydramine.

An antidepressant, includes, but is not limited to a selective serotonin reuptake inhibitor (SSRI), a tricyclic antidepressant, or another antidepressant drug, or a combination thereof.

A selective serotonin reuptake inhibitor (SSRI), includes, but is not limited to citalopram, fluoxetine, paroxetine, or sertaline, or a combination thereof.

A tricyclic antidepressant, includes, but is not limited to amitriptyline, imipramine, lofepramine, or nortriptyline, or a combination thereof.

Another antidepressant drug, includes, but is not limited to mirtrazapine, moclobemide, phenelzine, or venlafaxine, or a combination thereof.

Another aspect of the present invention includes a method as herein described further comprising deep brain stimulation and administering a therapeutically effective dose of tissue kallikrein for treating PD. Deep brain stimulation (DBS) is a surgical treatment in which electrodes (leads connected to the pulse generator) are implanted into areas of the brain responsible for symptoms of PD, for example, the subthalamic nucleus and the globus pallidus interna areas. DBS is commonly referred to as a brain pacemaker as the pulse generator sends electrical impulses deep into the brain along the implanted lead electrodes on a regular basis. DBS is primarily recommended to those who are treatment-resistant to PD drug therapies.

“Treatment” and “treating” refer to preventing, inhibiting, and/or alleviating Parkinson's disease or dementia with Lewy bodies, and related symptoms as well as healing Parkinson's disease or dementia with Lewy bodies, conditions or symptoms affecting mammalian organs and tissues. A composition of the present invention can be administered in a therapeutically effective amount to a patient before, during, and after any above-mentioned condition arises.

### Intranasal Administration

An aspect of the invention includes a composition formulated for intranasal administration comprising about 0.001 to about 5000 IU of KLKI, or a variant or an active fragment thereof, optionally comprising a pharmaceutically acceptable excipient. A composition can be administered to the nasal cavity of a human or other mammal to diseased areas of the brain by means of the olfactory neural pathway. The method may employ a pharmaceutical composition capable of transporting KLK1 to diseased neurons of the brain.

A method of the invention may achieve delivery of compounds to afflicted areas of the brain through transneuronal retrograde and anterograde transport mechanisms. Delivery of neurologic agents to the brain by that transport system may be achieved in several ways. One technique comprises delivering a neurologic agent alone to the nasal cavity. In this instance, chemical characteristics of KLK1 can facilitate its transport to diseased neurons in the brain. Alternatively, KLK1 may be combined with other substances that assist in transporting KLK1 to sites of damaged neurons. Auxiliary substances are capable of delivering KLK1 to peripheral sensory neurons and/or along neural pathways to dysfunctional areas of the brain. Peripheral nerve cells of the olfactory neural pathway can be utilized in order to deliver KLK1 to damaged neurons in those regions of the brain that are connected to the olfactory bulb.

KLK1 can be administered to the nasal cavity alone or in combination with a second therapeutic compound useful in treating PD or DLB. KLK1 can be combined with a carrier and/or other adjuvants to form a pharmaceutical composition. Potential adjuvants include, but are not limited to, GM-1, phosphatidylserine (PS), and emulsifiers such as polysorbate 80. Further supplementary substances include, but are not limited to, lipophilic substances such as gangliosides and phosphatidylserine (PS).

A method of the invention delivers KLK1 to the nasal cavity of a mammal. It is preferred that KLK1 be delivered to the olfactory area in the upper third of the nasal cavity and particularly to the olfactory epithelium in order to promote transport of the agent into the peripheral olfactory neurons rather than the capillaries within the respiratory epithelium. Transport of KLK1 to the brain by means of the nervous system instead of the circulatory system so that KLK1 can be delivered to damaged neurons in the brain.

In one embodiment of the method of the invention, KLK1 can be combined with micelles comprised of lipophilic substances. Such micelles may modify the permeability of the nasal membrane and enhance absorption of the agent. Lipophilic micelles can include gangliosides, particularly GM-1 ganglioside, and phosphatidylserine (PS).

Once KLK1 has crossed the nasal epithelium, the invention further provides for transport of KLK1 along the olfactory neural pathway. KLK1 may be capable of movement within the olfactory system. In particular, neurotrophic and neuritogenic substances have demonstrated ready incorporation into nerve cell membranes and an affinity for nerve cell receptor sites.

To deliver KLK1 to olfactory neurons, KLK1 alone or in combination with other substances as a pharmaceutical composition may be administered to the olfactory area located in the upper third of the nasal cavity. The composition may be dispensed intranasally as a powdered or liquid nasal spray, nose drops, a gel or ointment, through a tube or catheter, by syringe, by packtail, by pledget, or by submucosal infusion.

A pharmaceutical composition for intranasal administration may be formulated as a powder, granules, solution, ointment, cream, aerosol, powder, or drops. A solution may be sterile, isotonic or hypotonic, and otherwise suitable for administration by injection or other means. In addition to KLK1, a solution may contain appropriate adjuvants, buffers, preservatives and salts. Powder or granular forms of a pharmaceutical composition may be combined with a solution and with diluting, dispersing and/or surface active agents. Solutions such as nose drops may contain antioxidants, buffers, and the like.

Intranasal administration methods provide an advantage of the intranasal administration of the medication. The olfactory system provides a direct connection between the outside environment and the brain thus providing quick and ready delivery of KLK1 for treating PD and DLB. Moreover, means of applying a pharmaceutical composition intranasally can be in a variety of forms such as a powder, spray, or nose drops that obviates intravenous or intramuscular injections and simplifies administration of therapeutic medications.

The invention will be described with reference to various specific and preferred embodiments and techniques. However, it should be understood that many variations and modifications may be made while remaining within the spirit and scope of the invention.

All publications, patent applications, and issued patents cited in this specification are herein incorporated by reference as if each individual publication, patent application, or issued patent were specifically and individually indicated to be incorporated by reference.

### EXAMPLES

### Example 1

### In vitro Cleavage of α-Synuclein by KLK1

### Cleavage Assay

To assay the ability of KLK1 to cleave full length α-synuclein (1-140 AA), recombinant α-synuclein (rPeptide) was treated with porcine pancreas derived KLK1 in PBS solution. The following samples were incubated for 24 hours at 37° C.: 100 nM KLK1+2.5 μM α-synuclein, 100 nM KLK1 alone, and 2.5 μM α-synuclein alone. Each sample of KLK1 contained 50 nM of soybean trypsin inhibitor (Sigma, St. Louis). At the completion of the incubation period, the samples were run on a 15% SDS-PAGE gel which was then assayed by Western Blot on a PVDF membrane. The membrane was incubated in primary antibody (ab6162 or ab21975) diluted 1:5000 in blocking solution, followed by incubation with and alkaline phosphatase-conjugated secondary antibody. The membrane was then developed in a NBT-BCIP solution.

To assay the ability of KLK1 to cleave α-synuclein 1-95 (1-95 AA), recombinant α-synuclein 1-95 (GenWay Biotech) was treated with porcine pancreas derived KLK1 in PBS solution. The following samples were incubated for 3 hours at 37° C.: 1 mM KLK1 alone, 250 μg/ml α-synuclein 1-95 alone, and 1 mM KLK1 and 250 μg/ml α-synuclein 1-95. Each sample of KLK1 contains 100 μM of soybean trypsin inhibitor. At completion of the incubation period, the samples run on a 15% SDS-PAGE gel and which was then assayed by Western Blot on a PVDF membrane. The membrane was incubated in primary antibody (5C2) diluted 1:250 in blocking solution, followed by incubation with and alkaline phosphatase-conjugated secondary antibody. The membrane was then developed with chemiluminescent solution and exposed onto film.

### Analysis of Cleavage by Western Blot

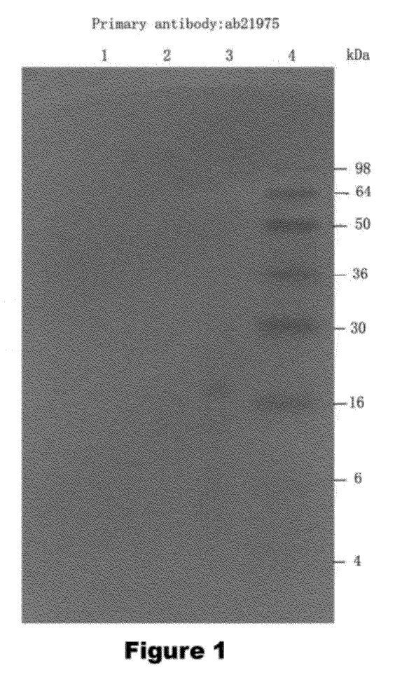

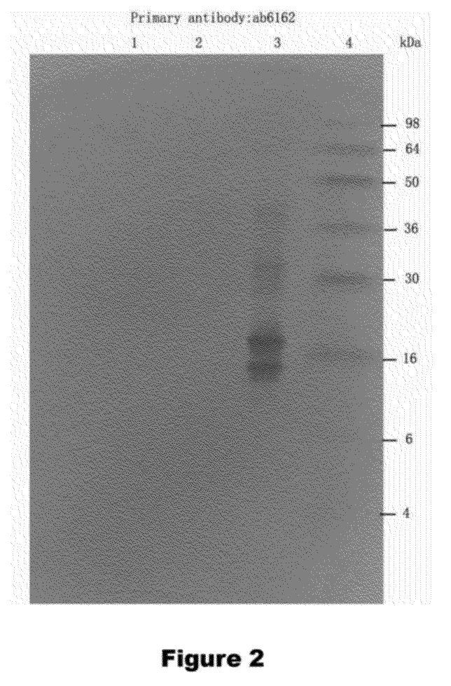

After 24 hours of treatment, all detectable α-synuclein was degraded by KLK1. α-synuclein was not detected after treatment with KLK1 by N-terminal specific (FIG. 1, lane 2) and C-terminal specific (FIG. 2, lane 2) antibodies, however could be detected as being intact when not treated with KLK1 (FIG. 1, lane 3 and FIG. 2, lane3). α-synuclein is the main component of neuronal protein aggregations called Lewy bodies and Lewy neurites. It is believed that these inclusions are neurotoxic and result in the death of doaminergic neurons within the substantia nigra of the brain in those with Parkinson's disease. α-synuclein degradation is disturbed in Parkinson's disease, and therefore treatment with KLK1 may prevent α-synuclein aggregation and formation of Lewy bodies and Lewy neurites.

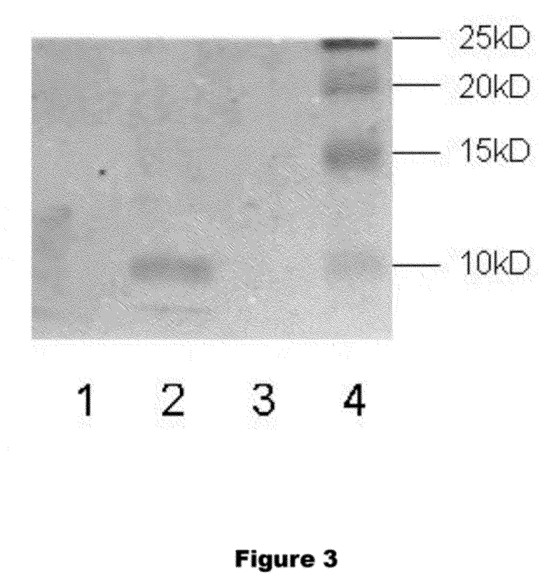

After 3 hours of treatment, all detectable α-synuclein 1-95 was degraded by KLK1. α-synuclein 1-95 was not detected after treatment with KLK1 by the non-β-amyloid component (NAC) specific antibody (FIG. 3, lane 3), but could be detected as being intact when not treated with KLK1 (FIG. 3, lane 2). The NAC region of α-synuclein is believed to be the aggregation domain due to its hydrophobic sequence, leading to the formation of Lewy body and Lewy neurite inclusions. The C-terminal sequence following NAC region appears to play an inhibitory role in preventing α-synuclein aggregation. C-terminal truncations with intact NAC regions of α-synuclein lead to enhanced aggregation, neurotoxicity of dopaminergic neurons and are found within Lewy inclusions of Parkinson's disease (Periquet et al, 2007). α-synuclein 1-95 is a C-terminal truncation with an intact NAC region, yet is completely degraded by KLK1, thereby suggesting KLK1 may be effective treatment against the most toxic form of α-synuclein in Parkinson's disease.

### Example 2

### Inhibition of GSK-3β by KLK1 Leads to Decreased α-Synuclein Expression and Cell Death from MPTP Treatment

The apoptosis of neurons leads to the neurodegeneration seen in Parkinson's disease. MPTP (N-methyl-4-phenyl-1,2,3,6-tetrahydropyridine) is a neurotoxin which imparts its toxic affects through the activation of the kinase GSK-36 which leads to increased expression of α-synuclein protein.

Treatment of Neuronal Cells with MPTP for 48 Hours

Primary mesencephalic neurons isolated from 16-18 day old rat embryos are plated onto 6 well dishes and grown in each well using Neurobasal medium (Invitrogen) supplemented with B27 (2% v/v, Invitrogen), penicillin/streptomycin mixture (10 U mL') and 25 μm β-mercaptoethanol at 37° C. and 5% CO₂. 50 μM of MPP⁺ iodide (Sigma, St. Louis, Mo.) is added to each well and allowed to incubate for 48 hours. In addition, KLK1 (0.01 to 100 Units) is added to the MPP⁺ treated wells 24 hours after the addition of the neurotoxin. A positive control is the non-treated well over 48 hours.

Western Blot Analysis after 48 Hours MPTP Treatment

The mesencephalic neurons are washed twice with ice-cold PBS and collected with 200 μl 2× stop solution (500 mM Tris-HCL, pH 6.8, 10% SDS, 100 mM EDTA, 100 mM EGTA, 10% glycerol) containing protease inhibitor mixture (Complete Mini, Roche Diagnostics GmbH, Germany) and the phosphatase inhibitor sodium orthovanadate (1 mM). The samples are resolved by NuPAGE 12% Bis-Tris (Invitrogen, Carlsbad, Calif.) SDS-PAGE gel electrophoresis and followed by electroblotting onto a PVDF membrane in NuPAGE transfer buffer. The membrane is then separately blotted using a rat specific α-Syn antibody (BD Transduction Laboratories, San Jose, Calif., 610786) for detection of full sized α-Syn and rat specific GSK-3β antibody (pY216, BD Transduction Laboratories, 612312). The quantization of the each treatment group lane is obtained by measuring optical density of each band and expressed as a ratio between α-Syn+β-actin, and GSK-β+p-GSK-3βY216, separately. The ratio values obtained for each treatment group lane are then compared to the control group (which is set to 100%).

### Results

MPTP leads to a significant increase in GSK-36 activation (increased pY216) and α-synuclein protein expression compared to the control. The Western blot shows that supplemental treatment of the neuronal cells 24 hours after MPTP treatment with KLK1 leads to a statistically significant decrease in GSK-3β activation (decreased pY216) and α-synuclein protein expression compared to MPTP treatment alone. These data suggest KLK1 can prevent the activation of GSK-3β and increased expression of α-Synuclein by MPTP.

### MTT Assay for Cell Viability

After 48 hours of treatment with MPTP, MPTP followed by KLK1 at 24 hours or non-treatment control, the mesencephalic neuron treatment groups are washed twice with D-PBS and then incubated for 2 hours with Neurobasal medium (Invitrogen) supplemented with B27 (2% v/v, Invitrogen), penicillin/streptomycin mixture (10 U mL¹), 25 μm β-mercaptoethanol and 0.5 mg mL⁻¹ of MTT (3-(4,5-dimethylthiazol-2-yl)-2,5-diphenyltetrazolium bromide, Sigma, St. Louis, Mo.) at 37° C. and 5% CO₂. The cells are then washed carefully twice with D-PBS and then the formazan salts are solubilized with pure ethanol. A sample from each treatment group is measured by absorbance at A=564 nm using a UV/V spectrophotometer using ethanol as the blank reference. The control absorbance reading is set to 100% and the other treatments are expressed as % of the control.

### Results

In comparison to the MPTP alone treatment group, the supplementation of KLK1 24 hours after MPTP results in no statistical difference in cell viability compared to the non-treatment control. This result suggests that KLK1 prevents the ability of MPTP to exert its toxicity on neuronal cells, likely through the ability of KLK1 to prevent the activation of GSK-3β and resulting increased α-synuclein expression.

### Example 3

### KLK1 Enhances Cell Viability after Treatment with α-Syn

### Aggregate Preparation:

Various forms of α-Syn (500 μM) (e.g. wild-type, minus C-terminal, A30P, A53T forms) are allowed to aggregate into fibrils by incubation in sterile water solution at 37° C. for three days, while non-aggregated forms are prepared immediately before application to the cultures.

### Cell Treatment:

At embryonic day 14, brain tissue is removed from fetal rats and the cells are plated (3×10⁵ cells per dish) in 96-well dishes (Corning, Corning, N.Y.) precoated with poly-d-lysine (50 mg/ml; Sigma, St. Louis, Mo.). The mesencephalic neurons are grown in minimum essential medium (Gibco Laboratories, Grand Island, N.Y.) supplemented with 15% fetal calf serum (Gibco) and glutamine (2 mM). Cultures are kept at 37° C. in a humidified 5% CO₂.

The cells are treated with aggregated and non-aggregated α-Syn (10, 25, 50, or 100 μM) on every second day for six days with or without KLK1 (0.01 to 100 Units) beginning 24 hours after cell plating. Using the MTT assay for cell viability (as per Example 2) the effect of α-Syn on mesencephalic neuron viability is determined, expressed as percentage of the non-treated control cell viability.

### Results:

The treatment of mesencephalic neurons with α-Syn leads to a significant decrease in cell viability compared to the non-treatment control group. The addition of KLK1 to the treatment with α-Syn leads to a significant increase in cell viability compared to the mesencephalic neurons treated with α-Syn only. This result shows the ability of KLK1 to protect mesencephalic neurons from α-Syn induced neurodegeneration.

### Example 4

### In vivo KLK1 Mediated Neuroprotection from MPTP Neurotoxin Treatment

MPTP is a neurotoxin which preferentially exerts its toxicity on dopaminergic neurons within the substantia nigra pars compacta (SNc), an area of the brain severely affected by neurodegeneration in Parkinson's disease.

### MPTP Treatment of C58BL/6N Mice

After a one week rest period, 12 week adult male C58BL/6N mice were divided into three treatment groups with 10 mice in each group. In the MPTP alone treatment group, mice are injected intraperitoneally (i.p.) with 30 mg/kg of MPTP (Sigma) daily for five consecutive days. Mice of the KLK1+MPTP treatment group are treated with intranasal administration of KLK1 (0.01 to 100 Units) one day before the start of MPTP i.p. administration and in the following 5 days thirty minutes before MPTP treatment. The control non-treatment group receives injections of saline solution for 5 days consecutively.

### Assessment of Neurodegeneration of the Substantia Nigra Pars Compacta

Seven days following the final injection of MPTP, midbrain slices are stained for tyrosine hydroxylase (TH⁺) activity of dopaminergic neurons by immunohistochemistry using an anti-mouse TH antibody (Chemicon) and DAB staining. TH⁺ cells within the substantia nigra pars compacta (SNc) are counted in a double blind manner determining the mean count of each right and left sides from four sections under a light microscope.

### Results:

In comparison to the saline vehicle treated control group, the MPTP treated group shows a statistically significant decrease in TH⁺ neurons. The KLK1+MPTP treatment group shows a statistically significant higher number of TH⁺ neurons within the SNc compared to MPTP alone group. KLK1 significantly prevents TH⁺ dopaminergic neuron death from MPTP mediated-apoptosis, such that KLK1 prevents neurodegeneration in the SNc, an area of the brain in PD in which neurodegeneration is commonly found.

### Assessment of Behavioural Impairment in MPTP-Treated Mice

10 days following the final injection of MPTP, mice in each treatment group are tested for behavioral impairment in a double blinded manner.

### Pole Test

Mice are placed on a vertical wooden pole (50 cm in length and 1 cm wide) with their head facing upwards. The total time required for a mouse to turn downward and climb to the ground is recorded, with a cut off of 120 sec.

### Catalepsy Test

Both forepaws of mice are placed on a horizontal bar elevated 15 cm above the ground (0.2 cm diameter). The total time a mouse maintained this position before lifting their hindpaws onto the bar is recorded.

### Swim Test

Mice are placed in water tubs (30 cm×20 cm×20 cm) at a water depth of 12 cm at 27° C. Swim scores are graded as follows: 0, hind part sinks with head floating; 1, occasional swimming using hind limbs while floating on one side; 2, occasional floating/swimming only; and 3, continuous swimming.

### Results

In comparison to the saline vehicle control group, the MPTP treated group exhibits poorer scoring in all three tests (longer time to descend, longer time to lift hind paws and lower swim test score). The KLK1+MPTP treated group exhibits statistically significant better scores in all three tests separately compared to the MPTP treated group. These results suggest that KLK1 attenuates behavioral impairments caused by MPTP, a neurodegeneration inducing neurotoxin.

### Example 5

### KLK1 Prevents the Reduction of Spontaneous Locomotion Observed in Truncated Human α-Synuclein (1-120) Transgenic Mouse

Transgenic mice expressing truncated human α-Synuclein (1-120) in an α-Syn null background under the TH promoter (Tofaris et al., J Neurosci. 2006, 26:3942-50) display the formation of Lewy bodies made of α-Syn in the SNc, much like the α-synucleinopathy seen in PD.

### Spontaneous Locomotor Activity Testing

Spontaneous locomotor activity (LMA) levels of the 18 month old transgenic mice compared to 18 month wild type mice are tested. The 18 month transgenic mice are subdivided into two groups of 20 each, in which one group is treated with KLK1 intranasally for eight weeks daily prior the start of LMA while the other is left untreated.

LMA testing is performed in clear Perspex® box (210×210×365 mm) cages fitted with two transverse infrared beams 10 mm from the base, spaced equally along the length of the box. The number of beam breaks during the 30 min session is recorded by a computer.

### Results

The 18 month old, non-treated transgenic mice show a statistically significant decrease in the number of infrared beam breaks compared to 18 month old wild-type mice. The 18 month old KLK1 treated transgenic mice show a significant increase in the number of infrared beam breaks compared to the 18 month non-treated transgenic group. This result suggests that treatment of transgenic mice expressing truncated α-Syn with KLK1 prevents the α-Syn mediated loss of locomotion as similarly seen in PD.

### Example 6

### Expression and Purification of Recombinant Human KLK1

To generate the recombinant human KLK1, a cDNA coding for pre-pro-human KLK1, the 262 amino acid residue sequence depicted in SEQ ID NO:3, was purchased from OriGene™ (Rockville, Md., USA). The KLK1 cDNA (Catalogue No. SC122623) is a human cDNA open reading frame clone, cloned into the multi-cloning site of OriGene's pCMV6-XL5 vector, between a cytomegalovirus (CMV) promoter to control transcription of cDNA coding for pre-pro-human KLK1 and a polyadenylation signal. This pre-pro-human KLK1 clone was sequenced and, using translation software, translated to reveal Seq ID No. 3. This sequence differed at 2 amino acid residues from the human KLK1 sequence in GenBank (Ref No. NP_{—}002248.1), which may be the result of single-nucleotide polymorphism or SNP's between individuals within a species. Specifically, the apparent SNP's in the pre-pro-human KLK1 resulted in an apparent E to Q at amino acid residue 145 of 262, and an apparent A to V position 188 of 262, as depicted in SEQ ID NO:3. Experiments were performed with pre-pro-human KLK1 having the amino acid sequence in SEQ ID NO.3.

The pre-pro-human KLK1 cDNA in the pCMV6-XL5 was transfected into a CHO cell line using the FreeStyle™ MAX CHO Expression System (Invitrogen, Carlsbad, Calif. Catalog no. K9000-20). The kit allowed for transient transfection of vectors into Chinese Hamster Ovary (CHO) cells, growth of the transfected CHO cells in 10 liter culture, and protein expression in defined, serum-free medium. The CHO cells are grown in suspension and transient transfection of the KLK1 vector was performed with the liposome reagent supplied in the kit as per instructions.

Expression and purification of recombinant human KLK1 were performed essentially as described by Hsieng S. Lu, et al, (Purification and Characterization of Human Tissue Prokallikrein and Kallikrein Isoforms Expressed in Chinese Hamster Ovary Cells, Protein Expression and Purification (1996), 8, 227-237). Briefly, following transfection and allowing sufficient time for expression of recombinant human KLK1, culture supernatant from the 10 liter culture of CHO cells was harvested by centrifugation followed by 0.2 ím filtration. Clarified supernatant was then concentrated, reacted with trypsin to activate the recombinant human KLK1. Because the transient transfection was performed with the cDNA coding for pre-pro-human KLK1, the recombinant human KLK1 secreted from the CHO cells was in an inactive proprotein form. Therefore, activity assay of cell culture supernatant KLK1 involves an activation step with trypsin digestion. Activation was performed with trypsin at 10 nM final concentration for 2 hours at room temperature, and the trypsin was inactivated with Soybean Trypsin Inhibitor.

Following activation of the recombinant human KLK1, ammonium sulphate was added to the supernatant, and it was loaded onto an Octyl Sepharose® column. The Octyl column elution pool of active KLK1 was further purified by Benzamidine affinity column. Pooled active fractions off the Benzamidine column were then buffer exchanged into DEAE equilibration buffer and polished by DEAE column. Active KLK1 fractions from DEAE were pooled and buffer exchanged into 1×PBS buffer. The final KLK1 bulk drug substance was aliquoted and stored at −20° C.

### Example 7

### Characterization of Purified Recombinant Human KLK1

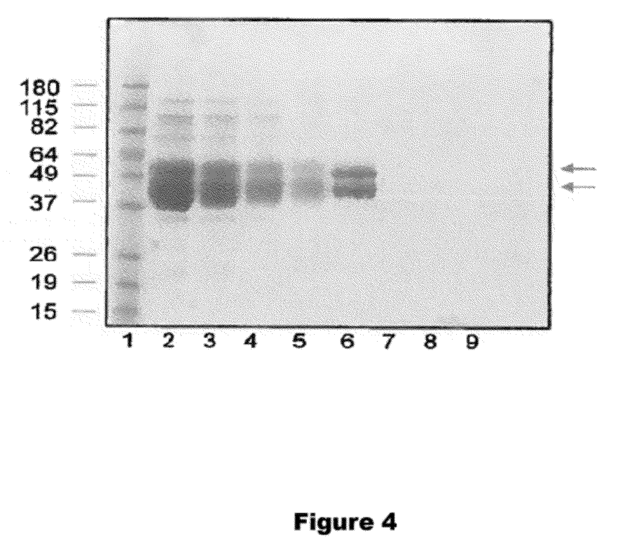

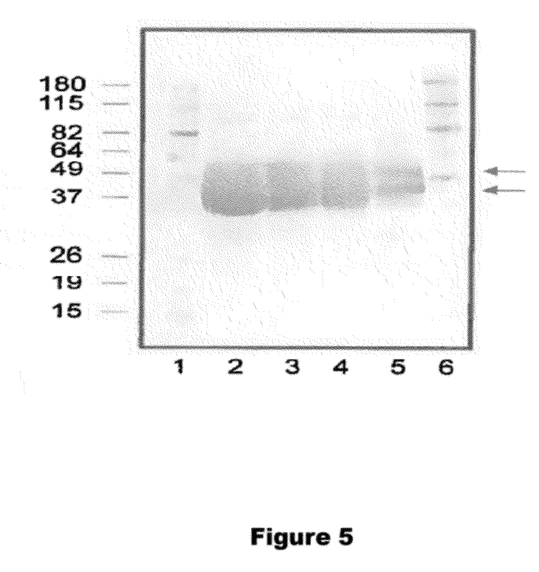

The purified recombinant human KLK1 contained approximately 30% carbohydrate content based on the molecular weight estimated by sodium dodecyl sulphate (SDS) polyacrylamide gel electrophoresis (see FIG. 4). KLK1 from CHO cells appears as a band having an apparent molecular weight of ˜40 to 49 kDa; such a broad band may result from different glycosylation forms of KLK1 secreted by CHO cells. For KLK1 expressed in 293 cells, two bands appeared on the SDS-PAGE gel at approximately 40 kDa and 45 kDa. The identity of the bands as human KLK1 was confirmed by Western blot analysis using mouse polyclonal antibody raised against a full-length human KLK1 protein (Catalog #: H00003816-B01P, KLK1 purified MaxPab mouse polyclonal antibody (B01P), Abnova Corporation, Walnut, Calif., USA) (see FIG. 5). The Western blot confirms the results of the SDS-PAGE gel, in that recombinant human KLK1 from CHO cells appears as a band having an apparent molecular weight of ˜40 to 49 kDa, and KLK1 expressed in 293 cells resolves as two bands at approximately 40 kDa and 45 kDa.

The purity of KLK1 from CHO was visually estimated from the SDS_PAGE gel to be >90% with a final concentration of 1.19 mg KLK1 protein/ml. From the SDS-PAGE gel, it appears CHO produced KLK1 also contains higher molecular weight impurities (˜70-95 kDa) that are not visible in the 293 preparation.

An enzyme activity assay was used to test for activity of recombinant human KLK1 in cell culture supernatants, chromatography fractions during purification and in the final purified product. One fluorogenic substrate suitable for tissue kallikrein measurement of activity is D-val-leu-arg-7 amido-4-trifluoromethylcoumarin (D-VLR-AFC, FW 597.6) (Sigma, Cat # V2888 or Ana Spec Inc Cat #24137.) When D-VLR-AFC is hydrolyzed, the free AFC produced in the reaction can be quantified by fluorometric detection (excitation 400 nm, emission 505 nm) or by spectrophotometric detection at 380 nm (extinction coefficient=12,600 at pH 7.2).

The measurement of recombinant human KLK1 activity (Units/ml) produced in the CHO cells was determined by comparing the relative activity of recombinant human KLK1 to the Kininogenase, Porcine standard acquired from the National Institute for Biological Standards and Control (NIBSC Product No. 78/543). For this standard, the assigned potency is 22.5 international units (IU) per 20 íg ampoule of porcine pancreatic kininogenase.

### Example 8

### Neuroprotective Activities of KLK1

The objective of this experiment was to determine if human recombinant KLK1 had neuroprotective activity in dopaminergic neurons in the primary, ventral mesencephalic culture. The ventral mesencephalic cell culture used in the study was a primary cell culture obtained from fetal rat brains. This cell culture is enriched with cells from the substantia nigra region of the brain where the dopaminergic neurons exist, and is also known as nigral cell culture.

The advantages of using this nigral cell culture system to test the effects of human recombinant KLK1 is the high percentage (20%) of dopaminergic (DAergic) neurons per field, which will increase the sensitivity of the assay for a test molecule, since small effects of different treatments are more likely to be resolved.

To isolate the primary, ventral mesencephalic culture, pregnant Sprague-Dawley rats were obtained from Harlan Laboratories, CA. On embryonic day 14, the pregnant rats were sacrificed and a laparotomy was performed to remove the uterine sacs. The fetal brain was isolated intact, and transferred to cold Leibovitz-15 (L-15) cell culture solution in a 15-cm petri dish. Next, the brainstem was isolated intact and transferred to fresh L-15 in a 15-cm petri dish. Finally, a 1.0 cubic mm piece of fetal brain tissue was isolated from the roof of the ventral mesencephalic flexure. From previous experience, 90% of nigral dopaminergic (DAergic) neurons are present in this piece of fetal brain tissue at this stage of development. The pieces of nigral brain tissue were collected in 5 ml of cold PCM-0 in a 15 ml Falcon tube. At the end of the dissection, the nigral tissue was washed (3×5 ml) with PCM-0, and suspended in 2 ml PCM-0. Tissue digestion was carried out with papain (1.0 U/ml; Worthington), for 15 min, 37° C. The digested tissue was washed (3×5 ml) with PCM-5 to inhibit papain protease activity. Finally the tissue was triturated in 2 ml PCM-5, and then spun (750 rpm, 2 min) in a Beckman bench top centrifuge, the supernatant aspirated and the pellet resuspended in 2.0 ml PCM-5. The cells were counted using a hemocytometer, and the final suspension density adjusted to 5.0×10₅ cells/ml with PCM-5. The cells were then plated as 25 μl droplets in the centre of wells of 8-well chamber slides previously coated with poly-D-lysine, and transferred to the incubator (37° C., 5% CO₂). Twelve hours later, PCM-5 was aspirated, replaced with PCM-0, and the drug treatment started.

Defined Primary Culture Medium (PCM): Composition

### 1. Advanced DMEM/F-12 (GIBCO, 12634)

2. Albumin, Fraction V, 1.0 mg/ml (Sigma, A-4161)
3. α-D-Glucose, 4.5 mg/ml (Sigma, 158968)

### 4. Glutamine 2.5 mM (Sigma, G-7513)

5. Insulin 5.0 ug/ml (Sigma, 1-1882)
6. apo-Transferrin 30 nM (Sigma, T-1428) ITS: I-1884
7. Sodium selenite 30 nM (Sigma, S-5613)

### 8. Progesterone, 20 nM (Sigma, P-6149)

### 9. Thyroxine, 30 nM (Sigma, T-0397)

10. Penicillin 50 U/ml and Streptomycin 50 μg/ml (Sigma, P-15140-148)
11. Fetal Bovine Serum (Hyclone FBS from Fisher: Cat No SH3007003) was also added and increasing concentrations are known to stimulate growth of neuronal cells. Different PCM medium have varying amounts of FBS, according to Table 1 below:

TABLE 1
PCM Media

| Medium code | Composition |
|---|---|
| PCM-0: | PCM + 0% FBS |
| PCM-2.5: | PCM + 2.5% FBS |
| PCM-5: | PCM + 5% FBS |
| PCM-10: | PCM + 10% FBS |

Additionally as positive control for protection of neuronal cells, medium conditioned by growing ventral mesencephalic cell line 1 (VMCL1) that produce factors that selectively protect nigral dopaminergic neurons. The medium thus conditioned is referred to as: ventral mesencephalic cell line 1 conditioned medium (VMCL1-CM) and the medium used as a positive control is 25% vol/vol ventral mesencephalic cell line 1-CM, (VMCL1-CM-25).

The composition of the ventral mesencephalic cell culture is described below (Table 2) and contained approximately 20% dopaminergic neurons.

TABLE 2
Neuronal Phenotypes in the Nigral, Ventral
Mesencephalic Cell Culture

| Phenotype | Percentage | Biomarker |
|---|---|---|
| Dopaminergic | 20 | Tyrosine hydroxylase (TH) |
| neurons | | Dopamine active transporter |
| | | (DAT) |
| GABAergic neurons | 50 | gamma-Aminobutyric acid |
| | | (GABA) |
| | | Glutamic acid decarboxylse |
| | | (GAD) |
| Serotonergic neurons | 7 | 5-hydroxytryptamine (5-HT) |
| Cholinergic neurons | 8 | Choline acetyltransferase |
| | | (ChAT) |
| Floor Plate (Non- | 10 | Sonic Hedgehog (SHH) |
| neuronal) | | |
| Astroblasts at plating | 2 | Vimentin |

Cellular composition of the culture: About 85% of the cells in the culture were neurons.

### Immunohistochemistry (IHC)

Immunohistochemistry was used to identify and differentiate the dopaminergic neurons from other neurons in ventral mesencephalic cell culture. Specifically, tyrosine hydroxylase (TH) was used as a marker for dopaminergic neurons, and microtubule associated protein-2 (MAP2) was used as a marker for neuronal cells in general.

At the end of the drug testing, the growth medium was aspirated, and the cells fixed, by adding 250 μl 4.0% paraformaldehyde (PFA) in 1×PBS per well at room temperature, for 15 minutes. Following fixation, aspiration and washing (1×PBS, 3×5 min), the cells were permeabilized by adding 250 μl cold 95% EtOH/1.0% glacial acetic acid per well for 10 min. After aspiration and washing: 1×PBS, 3×5 min; blocking was performed by adding 250 μl 5% normal horse serum (NHS) per well, for 1.0 hour, at room temperature. The blocking solution was aspirated, and 250 μl of the primary anti-TH mAb, diluted 1:5000 (Cat No. T2928-0.2ML; Sigma-Aldrich) in 5.0% NHS was added per well to odd numbered rows. The MAP2 mAb diluted 1:1000 (Cat No. M9942-200UL; SigmaAldrich) also diluted in 5% NHS, was added to even numbered rows. The antigen-antibody reactions took place over night, at 4° C. in the refrigerator. On day-2 of staining the TH and MAP2 primary antibodies were aspirated, and the cells washed: 1×PBS, 3×5 min. The secondary antibody, biotinylated anti-IgG Mo, raised in horse (Vector Labs), diluted 1:200, in 1.0% NHS, was added 250 μl per well, for 1 hr, at room temperature. Following aspiration and washing: 1×PBS, 3×5 min; 250 μl of the ABC complex for mouse (Cat No. PK-4002 Kit, Vector Labs) was added to each well. After aspiration and washing: 1×PBS, 3×5 min; 250 μl of the diaminobenzidine (DAB) substrate (Vector Labs) was added per well. The density of the dark color was allowed to develop, usually for 2-10 minutes, and monitored visually until the desired staining density was achieved. The reaction was stopped by adding 250 μl cold 1×PBS per well. At this stage, the chamber walls were removed. The stained cultures were dehydrated by serial passage through cell culture grade 95% EtOH (2×2 min), then 99.5% EtOH: 2×2 min. Finally the stained cultures were cleared by immersing in HistoClear (Cat No. HWS-200, National Diagnostics): 2×2 min. The stained microislands were then mounted in aqueous mounting medium (Cat No. H-5001, Vector Labs), and cover glasses applied. They were then dried in a fume hood for 3 hours, and sealed with clear nail polish, prior to analysis.

### Data Acquisition and Statistics

The cultures were analyzed using a Nikon TE2000U inverted microscope combined with the SimplePCI imaging software (Compix Inc, since acquired by the Hamamatsu Corp). Data were acquired using mainly a 20× objective, from 4-10 fields per data point, and the mean±SD determined. In a few instances, a 10× objective was also used. Statistical analysis was done using the SigmaPlot Statistics Program (V12), (Systat Software Inc.). An ANOVA analysis was first done to determine if there was any statistical difference among the members of a test group, usually >4 conditions. If no, then no further analysis was done, and the result of the experiment was reported as no statistical difference among the test groups. If yes, then multicomparisons of all logical data pairs were carried out, using either the Student-Newman-Keuls (SNK) test, or the Dunnett's test. Data are reported as vertical bar graphs, as Mean±SD (N).

### Daily Rating of Nigral Cell Cultures in Dose-Response Study to Human Recombinant KLK1

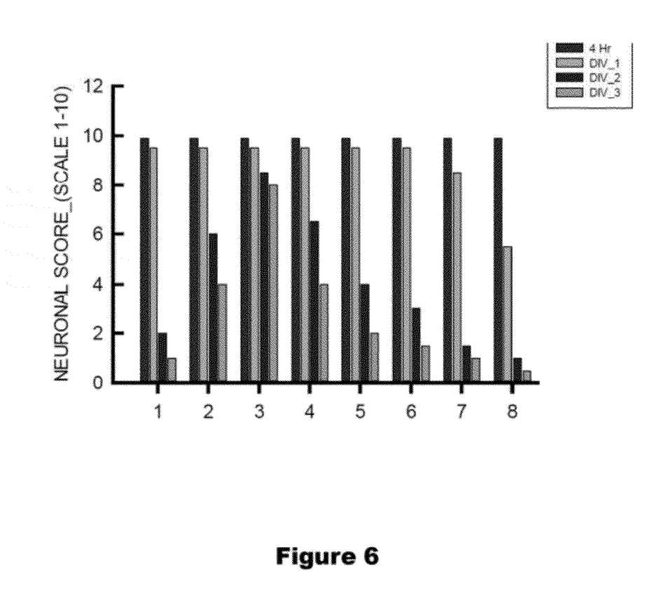

The nigral cell culture was established as described above from rat fetal brains. In order to obtain an historical record of the nigral cultures from plating to fixation prior to staining, the cultures are scored subjectively on a scale of 0-10, at intervals during the incubation period after serum withdrawal (PCM_{—}0% FBS). The results from neuronal counting after staining should coincide with the last subjective score. Twelve hours after plating, the PCM-5 media was aspirated and replaced with various culture conditions a (on the x-axis) 1: Negative control (PCN-0), 2: Positive control (cells grown in VMCL1-25 media), 3: Positive control (PCM-5), 4: PCN-0 plus hrKLK1 (10 U/ml), 5: PCN-0 plus Human recombinant KLK1 (hrKLK1) (5 U/ml), 6: PCN-0 plus hrKLK1 (1 U/ml), 7: PCN-0 plus hrKLK1 (0.2 U/ml), and 8: PCN-0 plus hrKLK1 (0.04 U/ml). The live dopaminergic cell cultures were scored at 4 h, and then every day after plating (DIV1—Day in vitro 1), DIV 2 and DIV3) on a scale of 1-10. The data are summarized in FIG. 6.

Results: On DIV2 and DIV3, the ratings for 5 and 10 U/ml hrKLK1 were better than the negative control at protecting nigral cell cultures from serum withdrawal, and at DIV3, 10 U/ml hrKLK1 appeared to be equivalent to the first positive control, VMCL1-25. The data were analyzed using the Kruskal-Wallis ANOVA on ranks, followed by multiple comparisons (Dunnett's Method) vs the Neg Con group.

### Testing the Response of Human Recombinant KLK1 on TH+ and MAP2+ Neurons in Nigral Cell Cultures

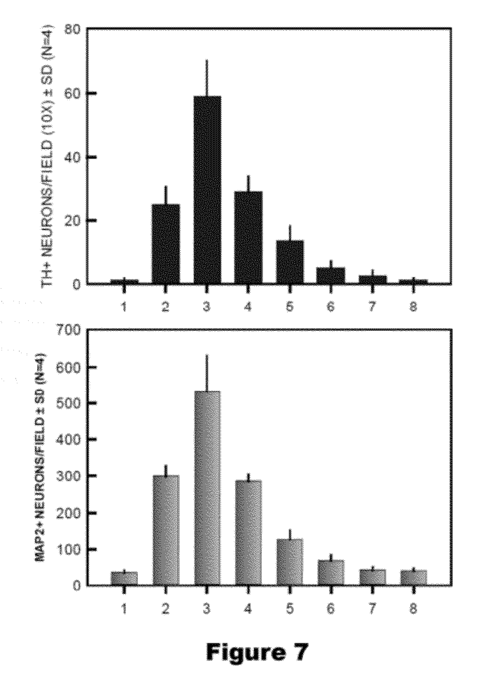

The cell cultures, described above, were analyzed by immunohistochemical (IHS) staining at (DIV3—Day in vitro 3 after serum withdrawal) and the numbers of TH+ and MAP2+ cells per field were determined. As previously described, tyrosine hydroxylase (TH) was used as a marker for dopaminergic neurons, and microtubule associated protein-2 (MAP2) was used as a marker for neuronal cells in general. The data are summarized on FIG. 7 (top graph: average TH+ neurons/field; bottom graph: average MAP2+ neurons/field). Statistics: Kruskal-Wallis ANOVA, H=29.2 (7); P<0.001. Dunnett's multiple comparisons: Pos Con PCM-5, Pos Con VMCL1, DM-199 10 U/ml vs Neg Con; for each, P<0.05.

Results: Treatment with hrKLK1 at 10 U/ml resulted in significant protection of TH+ (dopaminergic neurons) and MAP2+ (general neurons) compared to negative control after serum withdrawal at DIV 3 (see FIG. 7). The statistical results are identical for the TH and MAP2 data.

Testing Human Recombinant KLK1 on Protecting Nigral Culture from Neurotoxic Action of 6-OHDA

The ability of recombinant human KLK1 to protect neuronal cell cultures from the toxic effects of 6-hydroxydopamine (6-OHDA) was tested. As described above, twelve hours later after plating, PCM-5 was aspirated, replaced with PCM-0 and 6-OHDA (30 uM) for 5 minutes prior to KLK1 treatment being started.

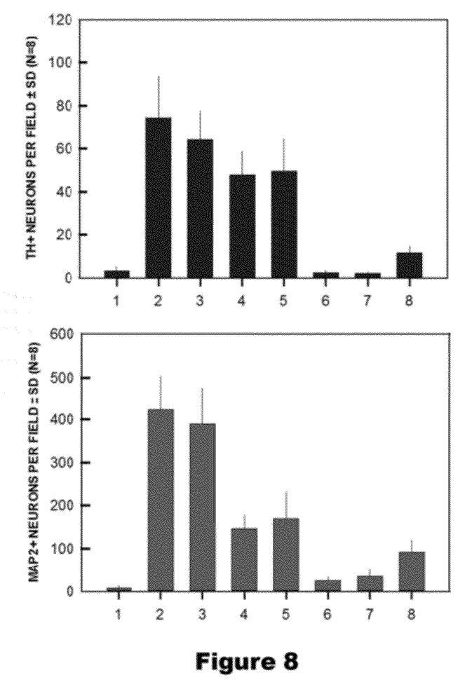

As shown in FIG. 8, 10 U/ml and 5 U/ml of recombinant human KLK1 protected DAergic (TH+) and the general neuronal population (MAP2+) (compare Lanes 3 and 4, to negative control in lane 1), which replicates the previous results.

Results: The addition of 30 um of 6-OHDA to the culture conditions results in the cell death of both DAergic (TH+) and the general neuronal population (MAP2+) (Lane 6). The addition of 5 U/ml of recombinant human KLK1 with 6-OHDA partially protected both TH+ and MAP2+ cells (Lane 8). Of the MAP2 neurons, a high percentage were dopaminergic (lane 8, TH+), suggesting KLK1 preferentially protects primarily dopaminergic neurons in the cell culture.

1. A method of treating Parkinson's disease or an associated condition comprising administering tissue kallikrein, or a variant or active fragment thereof.

2. The method of claim 1, comprising administering a polypeptide having at least 80% sequence identity to tissue kallikrein and having serine kinase activity.

3. The method of claim 1, further comprising administering an additional Parkinson's disease therapeutic compound.

4. The method of claim 3, wherein administering the additional Parkinson's disease therapeutic compound is concurrent with the administering of tissue kallikrein, or a variant or active fragment thereof, or polypeptide having at least 80% sequence identity to tissue kallikrein and having serine kinase activity.

5. A method of treating dementia with Lewy bodies or an associated condition comprising administering tissue kallikrein, or a variant or active fragment thereof.

6. The method of claim 5, comprising administering a polypeptide having at least 80% sequence identity to tissue kallikrein and having serine kinase activity.

7. A method of treating a disease or symptom selected from the group consisting of synucleinopathy, Parkinson-plus syndrome, dementia, delirium, visual hallucination parkinsonism, and depression, comprising administering tissue kallikrein or a variant or active fragment thereof, or a polypeptide having at least 80% sequence identity thereto and having serine kinase activity.

8. A composition comprising tissue kallikrein and a compound selected from the group consisting of a pharmaceutically acceptable carrier for intranasal administration, and a propellant for intranasal administration.

9. A composition comprising tissue kallikrein and an additional Parkinson's Disease therapeutic compound.

10. The composition of claim 9, where the additional Parkinson's Disease therapeutic compound is selected from the group consisting of an anticholinergic agent, an antiinfective agent, a catechol-O-methyl (COMT) transferase, a dopamine agonist, a monoamine oxidase type B (MAO-B) inhibitor, a neurological agent, a nutritional supplement, a psychotrophic agent, and an antidepressant.

11. The composition of claim 10 wherein the antiinfective agent is amantadine.

12. The composition of claim 10 wherein the COMT transferase is selected from the group consisting of carbidopa, entacapone, levodopa, and tolcapone.

13. The composition of claim 10 wherein the dopamine agonist is selected from the group consisting of apomorphine, bromocriptine, cabergoline, pergolide, pramipexole, and ropinirole.

14. The composition of claim 10 wherein the MAO-B inhibitor is selected from the group consisting of rasagiline and selegiline.

15. The composition of claim 10 wherein the neurological agent is selected from the group consisting of brasofensine, istradefylline, and leteprinim.

16. The composition of claim 10 wherein the nutritional supplement is selected from the group consisting of coenzyme Q-10, ubiquinone, and creatine.

17. The composition of claim 10 wherein the psychotrophic agent is diphenhydramine.

18. The composition of claim 10 wherein the antidepressant is selected from the group consisting of a selective serotonin reuptake inhibitors, a tricyclic antidepressant, mirtazapine, moclobemide, phenelzine, and venlafaxine.

19. The method of claim 1 further comprising deep brain stimulation.

20. The method of claim 1 wherein the tissue kallikrein is administered in a therapeutically effective amount.

21. The method of claim 20 wherein the therapeutically effective amount is 0.001 to 5000 IU per day, administered orally.

22. The method of claim 20 wherein the therapeutically effective amount is 0.001 to 5000 IU per day, administered intranasally.


SEQUENCE LISTING (3 sequences)

SEQ ID NO: 1 - protein, 263 aa, Sus scrofa
MWSLVMRLALSLAGTGAAPPIQSRIIGGRECEKDSHPWQVAIYHYSSFQCGGVLVDPKWVLTAAHCKNDNYQVWLGRHNLFENEVTAQFFGVTADFPHPGFNLSLLKNHTKADGKDYSHDLMLLRLQSPAKITDAVKVLELPTQEPELGSTCQASGWGSIEPGPDDFEFPDEIQCVELTLLQNTFCADAHPDKVTESMLCAGYLPGGKDTCMGDSGGPLICNGMWQGITSWGHTPCGSANKPSIYTKLIFYLDWINDTITENP

SEQ ID NO: 2 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

SEQ ID NO: 3 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTQEPEVGSTCLASGWGSIEPENFSFPDDLQCVDLKILPNDECKKVHVQKVTDFMLCVGHLEGGKDTCVGDSGGPLMCDGVLQGVTSWGYVPCGTPNKPSVAVRVLSYVKWIEDTIAENS